(19) United States
(12) Patent Application Publication
(10) Pub. No.: US 2013/0219925 A1
(43) Pub. Date: Aug. 29, 2013

(54) BLAST CHILLER APPARATUS AND A METHOD TO SANITIZE A BLAST CHILLER APPARATUS

(71) Applicant: Electrolux Professional S.p.A., (US)

(72) Inventors: Massimiliano Camatta, Porcia (IT); Massimo Pacorich, Cordovado (IT); Fabio Reale, Fontanafredda (IT); Fabio Sinatra, Cervignano del Friuli (IT)

(73) Assignee: ELECTROLUX PROFESSIONAL S.P.A., Pordenone (IT)

(21) Appl. No.: 13/771,862

(22) Filed: Feb. 20, 2013

(30) Foreign Application Priority Data
Feb. 29, 2012 (EP) 12157479.2

(51) Int. Cl. F25D 27/00; F25D 21/00

(52) U.S. Cl. CPC F25D 27/005; F25D 21/00
USPC 62/62; 62/264; 62/132

(57) ABSTRACT

Blast chiller apparatus (1) comprising a chilling chamber (3) structured for housing food to be cooled/frozen, a chilling circuit (6) comprising at least a first heat exchanger (8) designed to rapidly cool/freeze the chilling chamber (3); a defrost system (12) designed to defrost said at least first heat exchanger (8); at least an UV source (14) designed to irradiate ultraviolet radiations the inner space of said chilling chamber (3); and a control system configured to control the defrost system (12) to heat the first heat exchanger (8) so as to have the temperature of the chilling chamber (3) within a prefixed optimum temperature range of operating of said UV source (14).

The present invention relates to a blast chiller apparatus and a method to sanitize a blast chiller apparatus.

More specifically, the present invention relates to a blast chiller apparatus, which comprises a chilling chamber structured for housing food to be cooled, and is configured to quickly cool and/or freeze food arranged inside the chilling chamber, to which the following description refers purely by way of example without implying any loss of generality.

It should be pointed out that hereafter with the terms “sanitize”, it will be meant those factors that improve the general cleanliness of the chilling chamber of the chiller apparatus and aid to the preservation of health. In detail, the terms “sanitize” or “sanitization” as used herein means to free a targeted object from contaminants particularly in the form of living organisms such as the microorganisms.

As it is known, a most efficient way to “sanitize” an object, i.e. eliminate moulds, yeasts, bacterial spores and/or similar, consists in subjecting these micro-organisms to the action of ultraviolet radiations, as may be generated, for example, by UV sources/lamps currently available on the market.

One of the more common types of UV lamps that are used as sanitizing agents is the low pressure mercury lamp. Low pressure mercury lamps are generally cost effective in that their power requirements are low as compared to other types of UV light sources, yet low pressure mercury lamps also have a comparatively high UV output.

In view of the above advantages, UV mercury lamps have been proven to be effective in sanitizing the chilling chamber of blast chiller apparatus.

However, degradation in the performance of the UV mercury lamps operating inside of the chilling chamber is experienced due to the colder temperatures that fall well below the optimal operating temperatures of the UV mercury lamps.

As a matter of the fact, UV mercury lamps are unable to adequately function in cooling/freezing temperatures that, as it is known, fall outside optimal operating range of temperatures of the mercury lamps.

In order to perform the sanitizing cycle maintaining reasonable performance of the UV mercury lamps, in the blast chiller apparatus above disclosed, it is needed to open the door of the chilling chamber to cause the temperature inside of the chamber to grow up during a rest period of the chiller, so that the temperature inside of the chilling chamber reaches the outside temperature corresponding to the environment temperature.

When temperature inside the chilling chamber has reached the environment temperature, i.e. 15° C., the sanitizing cycle by UV lamp starts. Temperature reached inside of the chilling chamber in such conditions slightly improves the operating performance of UV mercury lamps.

However, environment temperature is usually lower than optimal operating range of temperatures, thus UV mercury lamps work with low efficiency, causing a partial degradation in the sanitizing performance.

Moreover, the sanitization operations need long time to be performed because, after opening the chiller-door, it is necessary to wait that temperature inside of cooling chamber reaches the environment temperature.

In addition, blast chiller apparatus above disclosed are typically provided with a chilling circuit comprising an evaporator which is arranged inside the chilling chamber and is structured to rapidly cool/freeze the latter.

However, the evaporator presents parts of its cooling surface which are shielded by means of an outer cover and thus are not irradiated from ultraviolet radiations, causing an incomplete sanitization of the microorganism that, as a consequence, remain on the evaporator shielded surfaces, affecting food preservability.

US 2005 0178 984 discloses a heat controlled ultraviolet light apparatus including a source of ultraviolet light, a cover, and a heating or cooling element that heats/cools the space between the ultraviolet light source and the cover.

Although ultraviolet light apparatus disclosed in US 2005 0178 984 is structured to optimize the output of the ultraviolet light source regardless of temperature at which the source is exposed during use, it is expensive and complex and is not able to irradiate microorganisms present on the evaporator shielded surfaces.

U.S. Pat. No. 6,237,250 discloses a refrigerated display case, including a goods platform, a goods space arranged above the platform, a tub space that is arranged below the platform and is bordered by a display case tub, a blower, an evaporating apparatus and a UV sterilizing tube. The evaporating apparatus is arranged in the effective range of the UV sterilizing tube, which admits the evaporating apparatus during the defrosting, carried out with the aid of a hot-gas defrosting device. During the relatively short defrosting phase, the temperature of the goods changes only insignificantly, that is to say by only 0.5 to 1° C.

Refrigerated display case disclosed in U.S. Pat. No. 6,237,250 is not able to sanitize completely all the evaporator surfaces.

In-depth research has been carried out by the applicant to achieve the following specific goals:

reduce sanitization time;

guarantee high performance of the UV light sources, in particular UV mercury lamps;

perform sanitization of the closed chilling chamber during an operating phase usually performed by the blast chiller apparatus, so that the system is simplified;

sanitize completely all the evaporator surfaces.

It is therefore an object of the present invention to provide a solution designed to achieve the above goals.

According to the present invention, there is provided a blast chiller apparatus comprising a chilling chamber structured for housing food to be cooled/frozen, a chilling circuit comprising at least a first heat exchanger designed to rapidly cool/freeze the chilling chamber, and a sanitizing system which is configured to sanitize said chilling chamber and comprises: a defrost system designed to defrost said at least first heat exchanger, at least an UV source designed to irradiate ultraviolet radiations the inner space of said chilling chamber; and a control system configured to control the defrost system to heat the first heat exchanger so as to have the temperature of the chilling chamber within a prefixed optimum temperature range of operating of said UV source.

Preferably the UV source comprises a UV-C mercury lamp designed to generate C-type ultraviolet radiations.

Preferably the control system is further configured to control the defrost system so as to have the temperature of the first heat exchanger within a prefixed sanitizing temperature range to cause a thermal sanitization of said heat exchanger.

Preferably, the control system is configured to control the defrost system to heat the first heat exchanger so as to have/maintain simultaneously the temperature of said first heat exchanger and the temperature of the chilling chamber in said prefixed sanitizing temperature range, and respectively in said prefixed optimum temperature range.

Preferably, the control system comprises temperature sensing means configured to measure temperature inside of said chilling chamber; the control system being further configured to switch-on said UV source to generate ultraviolet radiations when said measured temperature fall in said prefixed optimum temperature range.

Preferably the prefixed optimum temperature range is between about 30° C. and 50° C.

Preferably, the prefixed sanitizing temperature range is between about 50° C. and 80° C., more preferably between about 50° C. and 80° C.

Preferably the chilling circuit comprises a reversible-cycle heat pump assembly, which is provided with said first heat exchanger and is designed to work to perform a direct thermal cycle to rapidly cool/freeze the chilling chamber or, alternately, a reversed thermal cycle, to cause said first heat exchanger to be heated; during said reversed thermal cycle, said reversible-cycle heat pump assembly being said defrost system.

Preferably, the chilling circuit is provided with a heat pump assembly comprising refrigerant compressing means; said defrost system comprising a bypass channel which is interposed between the refrigerant-outlet of the refrigerant compressing means and the refrigerant-inlet of said first heat exchanger; and valve means arranged along said bypass channel for connecting, based on a command, the refrigerant-outlet of the refrigerant compressing means to the refrigerant-inlet of the first heat exchanger; said control system being configured to control said valve means to defrost said first heat exchanger.

Preferably, the defrost system comprise electric heating means associated with said first heat exchanger; said control system being configured to control said electric heating means to defrost said first heat exchanger.

According to the present invention, there is further provided a method to sanitize a blast chiller apparatus comprising a chilling chamber structured for housing food to be cooled/frozen, a chilling circuit comprising at least a first heat exchanger designed to rapidly cool/freeze the chilling chamber, a defrost system designed to defrost said at least first heat exchanger, and at least an UV source designed to irradiate ultraviolet radiations the inner space of said chilling chamber, the method comprising the step of controlling the defrost system to heat the first heat exchanger so as to have the temperature of the chilling chamber within a prefixed optimum temperature range of operating of said UV source.

Preferably the method comprises the step of controlling the defrost system (so so as to have the temperature of the first heat exchanger within a prefixed sanitizing temperature range to cause a thermal sanitization of said heat exchanger.

Preferably, the method comprises the step of controlling said defrost system to heat the first heat exchanger so as to have simultaneously the temperature of said first heat exchanger and the temperature of the chilling chamber in said prefixed sanitizing temperature range, and respectively in said prefixed optimum temperature range.

Preferably, the method comprises the steps of measuring temperature inside of chilling chamber; switching-on said UV source to generate ultraviolet radiations when said measured temperature fall in said prefixed optimum temperature range.

A non-limiting embodiment of the present invention will be described by way of example with reference to the accompanying drawings, in which:

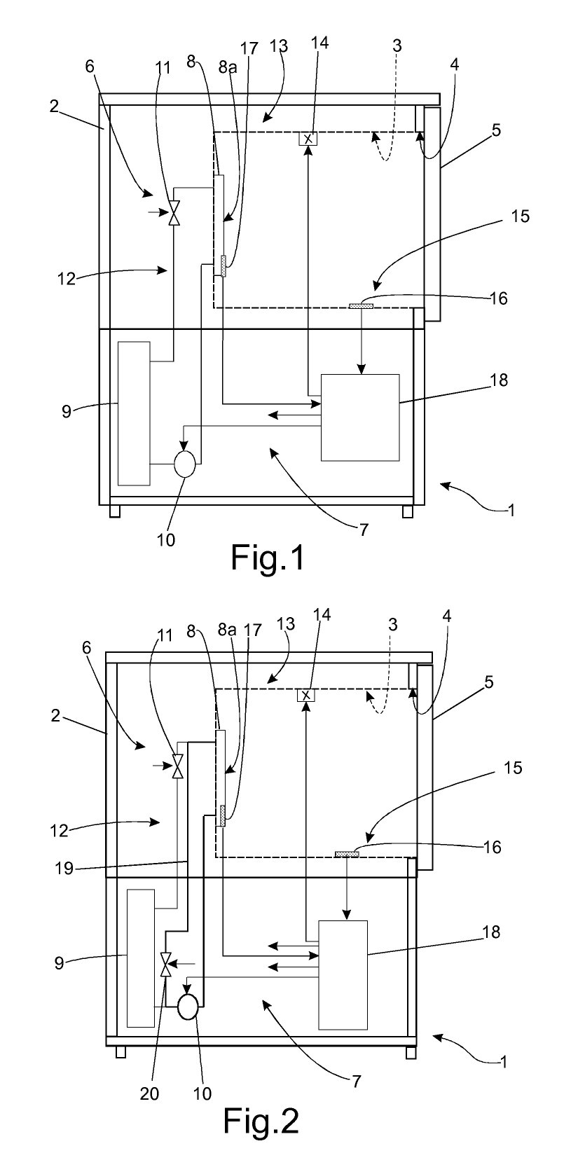

FIG. 1 shows a schematic lateral cross section of a blast chiller apparatus, according to the present invention;

FIG. 2 shows a schematic lateral cross section of a blast chiller apparatus, according to a first different embodiment of the present invention;

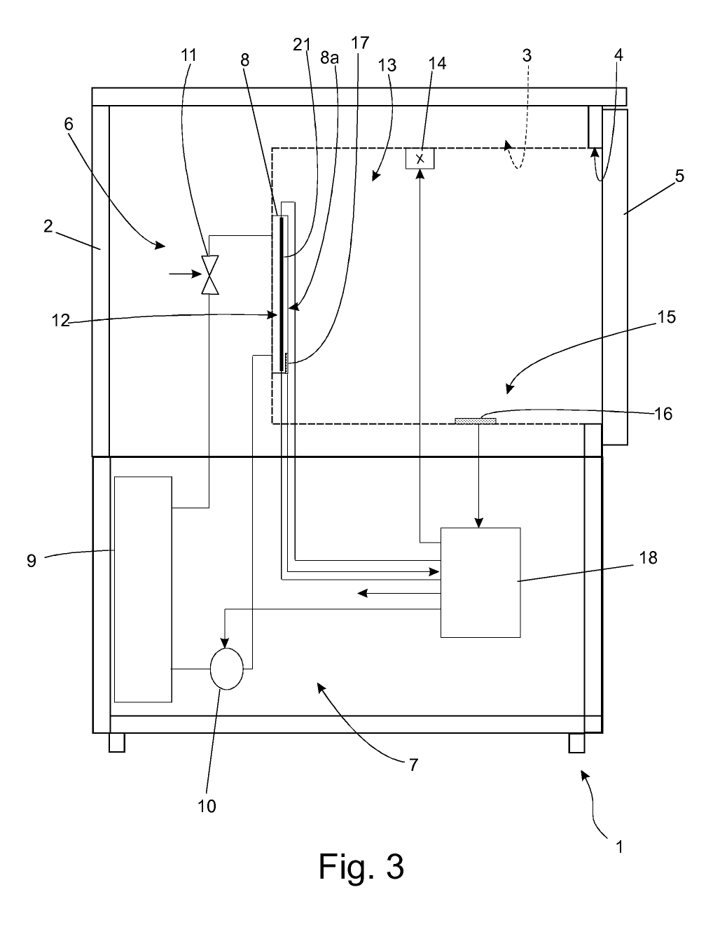

FIG. 3 shows a schematic lateral cross section of a blast chiller apparatus, according to a second different embodiment of the present invention.

With reference to accompanying Figure, referral number 1 indicates as a whole a blast refrigerating/freezing apparatus such as a professional or domestic/household blast chiller apparatus, which is structured to store food and is configured to quickly cooling or rapidly freezing the stored food.

It should be pointed out that hereafter with the terms “blast chiller apparatus”, it will be meant a professional or household blast chiller configured to bring the temperature of food, such as for example cooked food, from about +90° C./60° C. to about +3° C. in a short time, or a professional or household blast freezing chiller configured to bring the temperature of the food from about +90° C./60° C. to about −18° C. in a short time.

Referring to FIG. 1, blast chiller 1 comprises a preferably, though not necessarily, parallelepiped-shaped outer boxlike casing 2 structured for resting on the floor; a chilling chamber 3 which is structured for internally housing the food to be cooled, and which is located inside the outer casing 2, directly facing an access opening 4 preferably, though not necessarily, realized in the front wall of casing 2; and a porthole door 5 hinged to the front wall of casing 2 to rotate about a preferably, though not necessarily, vertically-oriented reference axis, to and from a closing position in which door 4 rests completely against the front wall to close the access opening 4.

According to a preferred embodiment of the present invention, the blast chiller apparatus 1 is also provided with a heat-pump type, chilling circuit 6 which is located inside the outer casing 2 and is structured to quickly cooling or rapidly freezing the chilling chamber 3.

Preferably, the heat-pump type, chilling circuit 6 is provided with a heat-pump assembly 7 comprising:

a first heat exchanger 8, which is located at least partially inside of the chilling chamber 3 and is designed so that the refrigerant fluid absorbs heat from air presents inside of the chilling chamber 3 in order to rapidly cooling down the food placed in the chamber 3;

a second heat exchanger 9, which is located outside of the chilling chamber 3 along the circulation refrigerant circuit of the heat-pump assembly 7, and which is designed to release heat absorbed by the refrigerant into the chilling chamber 3;

an electrically-powered refrigerant compressing device 10, which is interposed between the refrigerant-outlet of the first heat exchanger 8 and the refrigerant-inlet of the second heat exchanger 9, and which is structured for compressing the gaseous-state refrigerant directed towards the heat exchanger 9 so that refrigerant pressure and temperature are much higher at the refrigerant-inlet of second heat exchanger 9 than at the refrigerant-outlet of the first heat exchanger 8; and

an expansion valve 11 or similar passive/operated refrigerant expansion device (for example a capillary tube, a thermostatic valve or an electrically-controlled expansion valve) which is interposed between the refrigerant-outlet of the second heat exchanger 9 and the refrigerant-inlet of the first heat exchanger 8, and is structured so as to cause a rapid expansion of the refrigerant directed towards the first heat exchanger 8, so that refrigerant pressure and temperature are much higher at the refrigerant-outlet of the second heat exchanger 9 than at the refrigerant-inlet of the first heat exchanger 8.

The first heat exchanger 8, conventionally referred to as “evaporator” is structured so that the low-pressure and low-temperature refrigerant directed to the refrigerant compressing device 10 absorbs heat from the air present inside of the chilling chamber 3, thus causing cooling of the latter, while the second heat exchanger 9, conventionally referred to as “condenser”, is structured so that the high-temperature refrigerant arriving from the delivery of the refrigerant compressing device 10 releases heat to the environment. The refrigerant compressing device 10, whose function is clear from the above, is conventionally referred to as “compressor”.

According to a preferred embodiment of the present invention the heat-pump assembly 7 may be a “reversible-cycle heat pump” assembly designed to work either in a first operating state based on a direct thermal cycle, wherein the first heat exchanger 8 works as an “evaporator” in order to cool the chilling chamber 3 and the second heat exchanger 9 works as a “condenser” to release the absorbed heat, or in a second operating state based on a reversed thermal cycle, wherein the first heat exchanger 8 works as a “condenser” in order to release heat of the refrigerant to the cooling surfaces 8a of the first heat exchanger 8 to defrost the latter, and the second heat exchanger 9 operates as an “evaporator”.

According to FIG. 1, the blast chiller apparatus further comprises a sanitizing system 13 configured to sanitize the chilling chamber 3 and/or the evaporator's surfaces 8a faced to the inner space of the chilling chamber 3.

The sanitizing system 13 comprises a defrost system 12, which is designed to defrost the first heat exchanger 8, and at least a UV-source 14, which is preferably, though not necessarily, arranged inside of the chilling chamber 3 and is designed to generate ultraviolet radiation towards the inner surfaces of the chilling chamber 3 to be sanitized so that microorganisms are destroyed and/or inactivated.

Referring to the embodiment shown in FIG. 1, the defrost system 12 is defined by the heat-pump assembly 7 working in the second operating state, i.e. in the reversed thermal cycle, and is designed to defrost the cooling surfaces 8a of the first heat exchanger 8 faced to the inner space of the chilling chamber 3.

The UV-source 14 comprises preferably a UV-C mercury lamp designed to generate C-type ultraviolet radiations.

According to the present invention, the sanitizing system 13 further comprises an electronic control system 15 which is configured to control the defrost system 12 to heat the first heat exchanger 8 in order to have the temperature inside of the chilling chamber 3 within a prefixed optimum temperature range of operating of the UV source 14.

The electronic control system 15 is preferably further configured to control the defrost system 12 so that the temperature of the first heat exchanger 8 is within a prefixed sanitizing temperature range in order to cause a thermal sanitization of the first heat exchanger 8.

In detail, the electronic control system 15 is configured to control the defrost system 12 to defrost the first heat exchanger 8 and cause, during the defrost phase, the inner temperature of the chamber 3 to grow up until it reaches a first prefixed temperature associated with the prefixed optimum range of operating of the UV-source 14. The electronic control system 15 is preferably configured also to cause, during the defrost phase, the temperature of the first heat exchanger 8, in particular of its surfaces 8a, to grow up until it reaches a second prefixed temperature comprised in the prefixed sanitizing temperature. Moreover, the electronic control system 15 is configured to switch-on the UV source 14 to generate ultraviolet radiations, when said first and/or second prefixed temperatures are reached.

Preferably, the electronic control system 15 comprises a temperature sensing/measuring device 16 designed to measure the temperature inside of the chilling chamber 3, and/or a temperature sensing/measuring device 17 designed to measure the temperature of the surfaces 8a of the first heat exchanger 8. Moreover, the electronic control system 15 preferably comprises also an electronic control unit 18 which is configured to: receive in input the temperatures measured by the temperature measuring devices 16 and 17; operate the heat-pump assembly 7 to cause transition of the latter between the first and the second operating states, in particular from the first to the second operating state when the first heat exchanger 8 is to be defrosted; control the heat-pump assembly 7 during defrost phase based on the measured temperatures, so that the inner temperature of the chilling chamber 3 is raised up to the first prefixed temperature, and the temperature of the cooling surfaces 8a of the first heat exchanger 8 is raised up to the second prefixed temperature; and finally switch-on the UV source 14 when said first and/or second prefixed temperatures are reached.

The first prefixed temperature falls in the prefixed optimum temperature range of the UV-source 14, which is preferably between about 30° C. and 50° C. A suitable first prefixed temperature is for example about 40° C.

The second prefixed temperature associated with the prefixed sanitizing temperature range is preferably between about 50° C. and 80° C., more preferably between about 60° C. and 80° C. A suitable second prefixed temperature is for example about 70° C.

According to a different embodiment of the present invention shown in FIG. 2, the heat-pump assembly 7 comprises a bypass channel 19 connecting the refrigerant-outlet of the refrigerant compressing device 10 with the refrigerant-inlet of the first heat exchanger 8, and an electric valve 20, in particular a two-way valve, arranged along the bypass channel 19, and designed to operate, on command, between a closed state wherein it closes the bypass channel 19 to cause the heat-pump assembly 7 to operate in the first operating state above disclosed, and an open state, wherein the electric valve 20 connects the refrigerant-outlet of the compressing device 10 to the refrigerant-inlet of the first heat exchanger 8 so that the latter receives heated refrigerant compressed by the compressing device 10 and is subjected to defrost.

More specifically, the heated refrigerant causes defrost of the surfaces 8a of the first heat exchanger 8. As a consequences, the first heat exchanger 8, the refrigerant compressing device 10, the refrigerant channel connecting the refrigerant output of the first heat exchanger 8 to the refrigerant-input of the compressing device 10, the bypass channel 19 and the electric valve 20 define the defrost system 12.

According to the embodiment shown in FIG. 2, the electronic control unit 18 is configured to supply control signals to the electric valve 20 and to the expansion valve 11. When the first heat exchanger 8 is to be defrosted, the electronic control unit 18 sends control signals to close the expansion valve 11 and to open the electric valve 20. By opening the electric valve 20, the refrigerant-outlet of the compressing device 10 is connected to the refrigerant-inlet of the first heat exchanger 8 so that defrost of the latter, namely defrost of the evaporator, is started. The electronic control unit 18 is further configured to control the refrigerant compressing device 10 in order to cause, during defrost phase, raising of the inner temperature of the chamber 3 up to the first prefixed temperature and preferably also raising of the temperature of surfaces 8a up to the second prefixed temperature.

According to the different embodiment of the present invention shown in FIG. 3, the defrost system 12 comprises heating means 21 configured to heat the evaporator surfaces 8a in order to quickly defrost the latter.

Preferably, the heating means 21 may comprise at least an electric component, i.e. a resistor or similar, integrated with, coupled to, or arranged close to, the first heat exchanger 8 to cause defrost of the latter.

However, it should to be noted that heating means 21 may comprise any known system which is able to release heat to the evaporator surfaces 8a in order to cause defrosting of the latter.

In the embodiment of FIG. 3, the electronic control unit 18 is configured to control the operations of the heat-pump assembly 7, of the heating means 21 and of the UV source 14.

In particular, in order to sanitize the chilling chamber 3, the electronic control unit 18 is configured to switch off the heat-pump assembly 7; switch-on the heating means 21 to start defrosting, and to control, during the defrost phase, the heating means 21 (for example by performing a current-control, or voltage control, or power-control) based on the measured temperature(s), to raise the inner temperature of the chilling chamber 3 up to the first prefixed temperature and, preferably, also the surface temperature of evaporator surfaces 8a up to the second prefixed temperature. The electronic control unit 18 is also configured to switch-on the UV source 14 to generate ultraviolet radiations, when the first and/or second prefixed temperatures are reached.

Possibly, the defrosting technique according to FIG. 3, making use of heating means 21, can be used in combination with one of the techniques according to FIGS. 1 and 2.

The blast chiller apparatus according to the present invention has the major advantages of:

reducing sanitization time; as a matter of the fact, sanitization is made during defrost phase;

guarantying high performance of the UV light sources, because the latter works in the optimal operating range of temperatures of the mercury lamps;

being simple to be performed;

sanitizing completely all the evaporator surfaces; as a matter of the fact evaporator surfaces are subjected to a thermal sanitization treatment.

Clearly, changes may be made to the blast chiller apparatus and method as described and illustrated herein without, however, departing from the scope of the present invention.

1. A blast chiller apparatus (1) comprising a chilling chamber (3) structured for housing food to be cooled/frozen, a chilling circuit (6) comprising at least a first heat exchanger (8) designed to cool/freeze the chilling chamber (3);
characterized in comprising a sanitizing system (13) which is configured to sanitize said chilling chamber (3) and comprises:
a defrost system (12) designed to defrost said first heat exchanger (8);
at least an UV source (14) designed to irradiate with ultraviolet radiations the inner space of said chilling chamber (3); and
a control system (15) configured to control said defrost system (12) to heat the first heat exchanger (8) so as to have the temperature of the chilling chamber (3) within a prefixed optimum temperature range of operation of said UV source (14).

2. Blast chiller apparatus according to claim 1, wherein said control system (15) is further configured to control said defrost system (12) so as to have the temperature of the first heat exchanger (8) within a prefixed sanitizing temperature range to cause a thermal sanitization of said heat exchanger (8).

3. Blast chiller apparatus according to claim 1, wherein said control system (15) is configured to control the defrost system (12) to heat the first heat exchanger (8) so as to have or maintain simultaneously the temperature of said first heat exchanger (8) and the temperature of the chilling chamber (3) in said prefixed sanitizing temperature range and, respectively, in said prefixed optimum temperature range.

4. Blast chiller apparatus according to claim 1, wherein the control system (15) comprises at least a temperature sensor (16,17) configured to measure the temperature inside of said chilling chamber (3); the control system (15) being further configured to switch-on said UV source (14) to generate ultraviolet radiations when said measured temperature fall in said prefixed optimum temperature range.

5. Blast chiller apparatus according to claim 1, wherein said prefixed optimum temperature range is between about 30° C. and 50° C.

6. Blast chiller apparatus according to claim 1, wherein said prefixed sanitizing temperature range is between about 50° C. and 80° C.

7. Blast chiller apparatus according to claim 6, wherein said prefixed sanitizing temperature range is between about 60° C. and 80° C.

8. Blast chiller apparatus according to claim 1, wherein said chilling circuit (6) comprises a reversible-cycle heat pump assembly (7), which comprises said first heat exchanger (8) and is designed to work to perform a direct thermal cycle to cool said first heat exchanger (8) so as to rapidly cool/freeze the chilling chamber (3) or, alternately, a reversed thermal cycle, to heat said first heat exchanger (8), so as to defrost said first heat exchanger (8).

9. Blast chiller apparatus according to claim from 1, wherein said chilling circuit (6) is provided with a heat pump assembly (7) comprising refrigerant compressing means (10); a bypass channel (19) which is interposed between the refrigerant-outlet of the refrigerant compressing means (10) and the refrigerant-inlet of said first heat exchanger (8); and valve means (20) arranged along said bypass channel (19) for connecting, based on a command, the refrigerant-outlet of the refrigerant compressing means (10) to the refrigerant-inlet of the first heat exchanger (8); said control system (15) being configured to control said valve means (20) to defrost said first heat exchanger (8).

10. Blast chiller apparatus according to claim from 1, wherein said defrost system (12) comprises electric heating means (21) associated with said first heat exchanger (8); said control system being configured to control said electric heating means (21) to defrost said first heat exchanger (8).

11. Method to sanitize a blast chiller apparatus (1) comprising a chilling chamber (3) structured for housing food to be cooled/frozen, a chilling circuit (6) comprising at least a first heat exchanger (8) designed to rapidly cool/freeze the chilling chamber (3); a defrost system (12) designed to defrost said at least first heat exchanger (8); and at least an UV source (14) designed to irradiate with ultraviolet radiations the inner space of said chilling chamber (3); the method comprising the step of controlling the defrost system (12) to heat the first heat exchanger (8) so as to have the temperature of the chilling chamber (3) within a prefixed optimum temperature range of operating of said UV source (14).

12. Method according to claim 11, comprising the step of controlling the defrost system (12) so as to have the temperature of the first heat exchanger (8) within a prefixed sanitizing temperature range to cause a thermal sanitization of said heat exchanger (8).

13. Method according to claim 11, comprising the step of controlling said defrost system (12) to heat the first heat exchanger (8) so as to have simultaneously the temperature of said first heat exchanger (8) and the temperature of the chilling chamber (3) in said prefixed sanitizing temperature range, and respectively in said prefixed optimum temperature range.

14. Method according to claim from 11, comprising the steps of:
measuring the temperature inside of said chilling chamber (3);
switching-on said UV source (14) to generate ultraviolet radiations when said measured temperature falls in said prefixed optimum temperature range.

15. Method according to claim from 11, wherein said prefixed optimum temperature range is between about 30° C. and 50° C. and/or said prefixed sanitizing temperature range is between about 60° C. and 80° C.
